(19) United States
(12) Patent Application Publication
(10) Pub. No.: US 2010/0152819 A1
(43) Pub. Date: Jun. 17, 2010

(54) METHOD FOR ENHANCING BLOOD AND LYMPH FLOW IN THE EXTREMITIES

(75) Inventor: Kenneth J. MCLEOD, Vestal, NY (US)

Correspondence Address: NIXON PEABODY LLP - PATENT GROUP, 1100 CLINTON SQUARE, ROCHESTER, NY, 14604, US

(73) Assignee: Research Foundation of State University of New York, Binghamton, NY (US)

(21) Appl. No.: 12/571,835

(22) Filed: Oct. 1, 2009

Related U.S. Application Data
(62) Division of application No. 10/994,694, filed on Nov. 22, 2004.

(51) Int. Cl. A61N 1/04

(52) U.S. Cl. 607/76

(57) ABSTRACT

Methods for enhancing blood and lymph flow in the extremities of a human subject are disclosed. In one aspect, the methods rely on a stimulus effective to displace the skin of a plantar or palmer surface of the subject, thereby enhancing blood and lymph flow in the extremity associated with the stimulated plantar or palmer surface. In another aspect, the methods rely on an electrical stimulus to directly stimulate cutaneous receptors in a plantar or palmer surface of the subject, thereby enhancing blood and lymph flow in the extremity associated with the stimulated cutaneous receptors. Apparatus for enhancing blood and lymph flow in the extremities of a human subject according to the methods of the present invention are also disclosed.

This application is a divisional of U.S. patent application Ser. No. 10/994,694, filed Nov. 22, 2004, which is hereby incorporated by reference in its entirety.

This invention was made with government support under grant number 1RO1HL66007 awarded by the National Heart Lung and Blood Institute of the National Institutes of Health. The government has certain rights in this invention.

### FIELD OF THE INVENTION

The present invention relates to methods for enhancing blood and lymph flow in the lower body and upper extremeties of a human subject.

### BACKGROUND OF THE INVENTION

Poor blood flow and fluid flow in the extremities results in numerous serious clinical conditions. In diabetics, reduced blood flow (“arterial insufficiency”) results in ulcerations, reduced wound healing ability, and neuropathies, commonly leading to amputation. Venous insufficiency (poor venous blood return) leads to edema and poor wound healing. Lymphatic insufficiency (poor lymphatic return) commonly leads to infection and associated complications. Pooling of blood in the limbs during upright posture leads to a condition referred to as orthostatic intolerance (“OI”), resulting in a depressed blood pressure and increased heart rate or tachycardia. Complications of OI include, chronic fatigue syndrome, lower back pain, fuzzy vision, poor concentration, and loss of balance/dizziness. Perhaps the most serious complication of reduced blood flow in the extremities, however, is stasis in the lower limbs, and in particular, the deep femoral veins, which can lead to the condition of deep vein thrombosis (“DVT”). Emboli from such thrombi can break away and lodge in the lungs, often leading to death. While DVT has long been known to be a serious complication of surgery, it has recently been recognized to arise from extended immobility, such as that associated with extended bed rest, long distance travel, and other similar stationary circumstances. An additional complication of blood and lymph stasis is a reduced ability of tissues to adapt and heal. Blood stasis can lead to ulceration of tissue and/or failure of skin ulcers to heal, leading to significant complications. Pressure sores and ulcers are a common complications associated with extended immobility, including sitting and bedrest. In addition, poor venous and lymphatic return result in the buildup of fluid pressures in the limbs, depriving the bone tissue of necessary nutrient flow, resulting in bone loss (osteoporosis), and as well exacerbates the complications associated with impaired blood flow common in diabetes.

Thus, prevention of blood and lymph stasis is recognized as a critical problem in medicine, home healthcare, long distance travel, and increasingly, even in the workplace. Convenient means to enhance blood and lymph flow are not currently available.

Current commercialized technologies available for enhancing blood and fluid return from the limbs include passive devices such as support stockings (which reduce venous capacity), and active devices such as pneumatic compression and sequential pneumatic compression devices, which serve to forcibly extrude blood and fluid from an extremity, through low frequency periodic compression of the tissue. In addition, numerous technologies based on vibrating the body, or parts, thereof, have been proposed over the years.

U.S. Pat. No. 6,620,117 to Johnson et al., for example, describes an apparatus and method for imparting whole body vibration, in a horizontal, orbital motion, to an individual standing on the apparatus platform. U.S. Pat. No. 3,077,869 to Houbeau et al. and U.S. Pat. No. 4,782,822 to Ricken, describe various apparatus for inducing whole body vibration to a standing individual. Houbeau applies high frequency vibrations of 4000-6000 Hz. U.S. Pat. Nos. 1,886,452 and 1,899,544 to Whitney and U.S. Pat. No. 2,448,162 to Wettlaufer, describe apparatus to vibrate the entire body in a supine position, via vibrations or undulations applied to the back or thoracic region of a subject. U.S. Pat. Nos. 5,191,880 and 5,273,028 to McLeod et al., describe methods and apparatus for imparting whole body vibration to induce loading of the skeletal system at up to 50-500 microstrain. Whole body exposure to vibrations of 1.6 to 4.6 g have been shown to increase muscle blood flow in a standing patient by 20%. Zhang et al., “Blood Flow in the Tibialis Anterior Muscle by Photoplethysmography During Foot-Transmitted Vibration,” Eur. J. Appl. Physiol. 90:464-469 (2003). Current international standards, however, preclude exposing humans to accelerations of this level for more than a few seconds on a daily basis. In the treatment of DVT, for example, patients typically require treatment for 20 or more hours per day, for weeks at a time. In addition, it has been reported, that the application of continuous whole body vibration can, in fact be detrimental to lower body blood flow. Dreszer et al., “Effect of Vibration on the Functioning of the Lymphatic System in the Small Intestine in Rat,” Med. Pr. 30:331-336 (1980).

U.S. Pat. No. 2,645,219 to Bertholin provides a vibrating machine for imparting vibrations to entire limbs. U.S. Pat. No. 3,035,570 to Nelson and U.S. Pat. No. 3,370,584 to Girten, describe apparatus for providing lateral or oscillating movement to the feet relative to the ankle or leg.

Thus, not only are existing methods and devices uncomfortable, but their chronic use can be irritating to the skin and underlying tissue, and even organ systems, thus decreasing patient compliance and precluding long term use. In addition, continuous use of prior art methods and apparatus that rely on whole body vibration can, in fact, have a deleterious effect on lower body circulation.

The present invention is directed to overcoming these and other deficiencies in the art.

### SUMMARY OF THE INVENTION

One aspect of the present invention relates to a method of enhancing blood and lymph flow in the lower body of a human subject, which involves applying a stimulus to a plantar surface in the lower body of the subject. The stimulus is effective to displace the skin of the plantar surface between about 10 and 100 microns in amplitude, thereby enhancing the blood and lymph flow in the lower body associated with the stimulated plantar surface.

Another aspect of the present invention relates to a method of enhancing blood and lymph flow in the upper extremeties of a human subject, which involves applying a stimulus to a palmer surface of the subject. The stimulus is effective to displace the skin of the palmer surface between about 10 and 100 microns in amplitude, thereby enhancing the blood and lymph flow in the extremities associated with the stimulated palmer surface.

A further aspect of the present invention relates to a method of enhancing blood and lymph flow in the extremities of a human subject, which involves applying a stimulus to one or more plantar or palmer surfaces in an extremity of the subject. The stimulus is effective to displace the skin of the plantar or palmer surface between about 10 and 100 microns in amplitude, thereby enhancing the blood and lymph flow in the extremity associated with the stimulated plantar or palmer surface.

Yet another aspect of the present invention relates to a method of enhancing blood and lymph flow in the extremities of a human subject, which involves applying an electrical stimulus to one or more plantar or palmer surfaces in an extremity of the subject. The stimulus is effective to directly stimulate cutaneous receptors in the plantar or palmer surface, thereby enhancing blood and lymph flow in the extremity associated with the stimulated cutaneous receptors.

According to the present invention, plantar and palmer stimulation using electrical, or low level oscillatory, or relatively high frequency vibrational stimulation, requires no connection to the body. Simple contact of the plantar or palmer surface with the stimuli surface is sufficient to trigger somata-sensory detection, with subsequent coupling to the musculature through reflect arcs and central nervous system pathways. Blood and fluid return from the limbs is therefore accomplished through normal skeletal pumping mechanisms. As the required stimulus to trigger this response is minimal, long term use can be prescribed without concern of complications. Moreover, as no “set-up” per se is required for use of the invention, compliance with treatment is much more readily accepted by the user.

Considering applications to deep vein thrombosis (DVT) alone, the present invention can result in a significant health care costs savings. Orthopaedic surgery, and in particular joint replacement surgery, and the chemotherapy techniques associated with treatment of cancer represent two medical situations where incidence of deep vein thrombosis is remarkably common, though all forms of extended surgery contribute to the incidence rate. 1-2% of patients developing a deep vein thrombosis die from a pulmonary embolism resulting from a DVT, while the remaining patients will incur costs of close to $10,000 per incidence. Over 1,000,000 individuals a year are hospitalized for DVT in the U.S. at a cost exceeding $10B per year. The present invention represents a relatively low cost approach to significantly reducing or eliminating many of these complications.

### BRIEF DESCRIPTION OF THE DRAWINGS

FIGS. 1A-1C are graphic representations of supine measurements made during venous occlusion plethysmography. FIG. 1A shows a typical experiment in which blood flow is measured in triplicate by venous occlusion followed by incremental occlusions using 10 mmHg steps to determine the volume-pressure relation. FIG. 1B depicts the derivation of limb blood flow by fitting a straight line to the initial portion of the occlusion curve. FIG. 1C depicts the means by which volume changes during pressure steps can be partitioned into contributions from venous filling and microvascular filtration

FIG. 2 is a graphic representation of the microvascular filtration relation—the fitted linear relation between limb filtration flow and occlusion cuff pressure. Filtration occurs only above a critical occlusion pressure, Pᵢ. The slope is K_{f}, the microvascular filtration coefficient. By extrapolation the y-intercept, or the normalized filtered flow at zero hydraulic pressure, may be obtained which is related to interstitial pressures, oncotic pressure and lymphatic drainage.

FIGS. 3A and 3B are IPG tracings. FIG. 3A shows AZ as a function of time, and FIG. 3B shows ∂Z/∂t versus time. Ejection time T and ∂Z/∂tₘₐₓ are indicated. Blood flow is calculated as [HR·ρ·L²·T·∂Z/∂tmax ]/Z₀²

FIG. 4 is a graphic representation of the effect of plantar vibration on the volume-pressure capacitance relation. The relation displayed is obtained by normalizing all data to the maximum capacity obtained for an individual patient. Systematic deviations are detectable as a shift in the curve. 0 Hz represents no vibration, 15 Hz plantar vibration at 0.2 g, and 45 Hz plantar vibration at 0.2 g are shown. Plantar stimulation is shown to have no deleterious effect on venous capacitance.

FIG. 5 is a graphic representation of the alteration of the microvascular filtration relation as a result of plantar vibration. 0 Hz represents no vibration, and 45 Hz plantar vibration at 0.2 g (25 micrometers p-p) are shown. The slope, K_{f}, is not affected by the vibration stimulus. However, the absolute values of Pᵢ and the absolute value of the Y intercept, which reflect lymphatic flow, are significantly increased by the vibration. The figure is for supine data; upright data are similar.

### DETAILED DESCRIPTION OF THE INVENTION

The present invention provides methods and apparatus for enhancing blood and lymph flow in the lower and upper body extremities, i.e., the arms and or legs, of a human subject, though enhancement of skeletal muscle pump activity.

In one aspect, the present invention provides methods that involve applying a stimulus to at least one plantar or palmer surface of the subject, effective to displace the skin of the plantar or palmer surface between about 10 and 100 microns in amplitude.

The method of the present invention can be used to treat a variety of conditions, including edema, poor wound healing, lymphedema, orthostatic intolerance (hypotension when upright), chronic fatigue syndrome, lower back pain, fuzzy vision, poor concentration due to hypotension, loss of balance/dizziness/fainting, deep vein thrombosis, pulmonary embolism, skin ulcers, pressure sores, bone loss/osteoporosis, and blood flow related complications of diabetes.

The stimulus may, for example, be an oscillatory, vibrational, or mechanical stimulus. The vibrational stimulus is preferably at a frequency in the range of about 20 to 75 Hz, preferably in the range of about 30 to 60 Hz, and more preferably, about 45 Hz.

Apparatus useful in methods of the present invention include at least one support for a plantar or palmer surface of the subject, the support being adapted to provide a stimulus to the plantar or palmer surface effective to displace the skin of the plantar or palmer surface between about 10 and 100 microns in amplitude. The apparatus also includes a power source capable of providing the appropriate stimulus.

The apparatus may be adapted to provide an oscillatory stimulus, a vibrational stimulus, or a mechanical stimulus. Preferably, the apparatus is adapted to provide a vibrational stimulus at a frequency in the range of about 20 to 75 Hz, more preferably in the range of about 30 to 60 Hz, and more preferably, about 45 Hz.

The apparatus may be further adapted, for example, to include at least one support for a plantar surface of the subject, and at least one support for a palmer surface of the subject. The apparatus may include, for example, a support for one or both feet of the subject, a support for one or both hands of the subject, or any combination thereof.

The power source may, for example, be manual or electrical.

The apparatus may further include a means for supporting the subject in a supine, or a sitting position. The means for supporting the subject may, for example, support the subject in a fully supine position, a fully upright sitting position, or at any tilted angle there between.

In another aspect, the present invention provides methods that involve applying an electrical stimulus to at least one plantar or palmer surface of the subject, effective to directly stimulate cutaneous receptors in the plantar or palmer surface.

Induced electric field intensities in the dermis of 0.1 V/m to 10 V/m in a frequency range of 30-60 Hz, more preferably about 45 Hz, are sufficient to stimulate the cutaneous somatasensory receptors on the plamar and plantar surfaces.

Apparatus useful in this aspect of the present invention include at least one support for a plantar or palmer surface of the subject, the support being adapted to provide an electrical stimulus to the plantar or palmer surface effective to directly stimulate cutaneous receptors in the plantar or palmer surface. The apparatus also includes a power source capable of providing the appropriate stimulus.

The apparatus may be adapted, for example, to provide an electrical stimulus to a mobile subject. A fitted sock, stocking, shoe, or glove with embedded electrodes which contact the plantar or palmar surface when put onto the extremity, can be utilized to produce an adequate electric field in the cutaneous surface to stimulate the somatosensory receptors.

The apparatus may be further adapted, for example, to include at least one support for a plantar surface of the subject, and at least one support for a palmer surface of the subject. The apparatus may include, for example, a support for one or both feet of the subject, a support for one or both hands of the subject, or any combination thereof.

The power source may, for example, be electrical, with power obtained from a power main, or from a storage (battery) device, or from a combination of energy generation (e.g. photocells) and storage.

The power source may, for example, be pneumatic or hydraulic.

The power source may be regenerative, converting available mechanical vibration at one frequency into vibration at the preferred frequency of operation of the device.

The apparatus may further include a means for supporting the subject in a supine, or a sitting position. The means for supporting the subject may, for example, support the subject in a fully supine position, a fully upright sitting position, or at any tilted angle therebetween.

According to the present invention, and in contrast to prior art methods, it is not necessary to vibrate the whole body, the musculo-skeletal system, the leg, nor even the foot, to achieve significant increases in blood and lymphatic flow in the lower limbs. The present invention instead relies on the application of an oscillating or vibrational stimulus to displace the skin, particularly on the glaborous surface of the foot, or the palmer surface of the hands, or of an electrical stimulus to directly stimulate cutaneous receptors in the plantar or palmer surfaces. In addition, methods of the present invention, which rely on frequencies of between about 20 and 75 Hz, require stimulus levels of about 100 microns or less, which is an advantage in that continuous exposure to greater stimulus levels at these frequencies can, in fact, be detrimental.

The present invention thus recognizes that plantar or palmer based stimulation influences skeletal tissue through its effects on peripheral blood flow and lymph flow. The invention is further described by reference to the following examples, where vibrational stimulation of the plantar surface was combined with upright tilt table testing, while examining blood flow and fluid flow parameters in the lower extremities. The examples are provided by way of illustration, not of limitation.

### Examples

### Example 1

### Patient and Control Subject Screening

Screening was conducted of consecutive female patients aged 45-70 years who were enrolled in a general internal medicine practice. Patients were excluded with a current fracture of the lower appendicular or axial skeleton, or history of back pain which could be exacerbated by the vibration protocol (see below), known peripheral vascular disease, peripheral neuropathy, uncontrolled hypertension (blood pressure exceeding 150 mm Hg or diastolic blood pressure 95 mm Hg despite treatment), congestive heart failure, diabetes, liver or kidney failure, hyperparathyroidism, multiple myeloma, metastatic carcinoma, Cushing's syndrome, collagen vascular disease, chronic angioedema or lymphedema, uncontrolled hyperthyroidism, chronic substance abuse, or any condition precluding the subject following the protocol or providing informed consent. Subjects with excessive alcohol use (>2 drinks/day) or who smoked were also excluded.

### Example 2

### Laboratory Evaluation

All experiments started at 9 AM after a brief fast (2 hours). The right arm and right calf blood pressure were monitored intermittently by oscillometry. A vibrating plate (see below) was placed on the footboard of an electrically driven tilt table (Cardiosystems 600, Dallas, Tex., USA). Patients wore rubber soled shoes to ensure electrical isolation and were asked to lie supine with their feet flush with the plate which initially was not oscillating. This situation was designated “0 Hz”. Patients were instrumented to measure blood flow by two forms of measurement: mercury in silastic strain gauge plethysmography (“SGP”) with venous occlusion congestion cuffs and impedance plethysmography (“IPG”). Occlusion cuffs were placed around the lower limb 10 cm above a strain gauge of appropriate size attached to a Whitney-type SGP. Ag/AgCl EKG electrodes for IPG were attached to the left foot and left hand which served as current injectors, and in pairs representing anatomic segments as follows: ankle to upper calf just below the knee (the calf segment), knee to iliac crest (pelvic and upper leg segment), iliac crest to midline xyphoid process (the splanchnic segment), and midline xyphoid process to supraclavicular area (the thoracic segment). Output from the strain gauge and impedance leads were interfaced to a personal computer through an A/D converter with a sampling rate of 200 samples per second per channel. (DataQ Ind, Milwaukee, Wis., USA). Data were multiplexed and effectively synchronized.

Subjects had vascular measurements made with plantar stimulation at 0, 15, and 45 Hz with the three frequencies presented in random order for a given subject. At each vibrational frequency, measurements were made supine and at 35° upright tilt.

### Example 3

### Peripheral Vascular Evaluation by Strain Gauge Plethysmography (“SGP”)

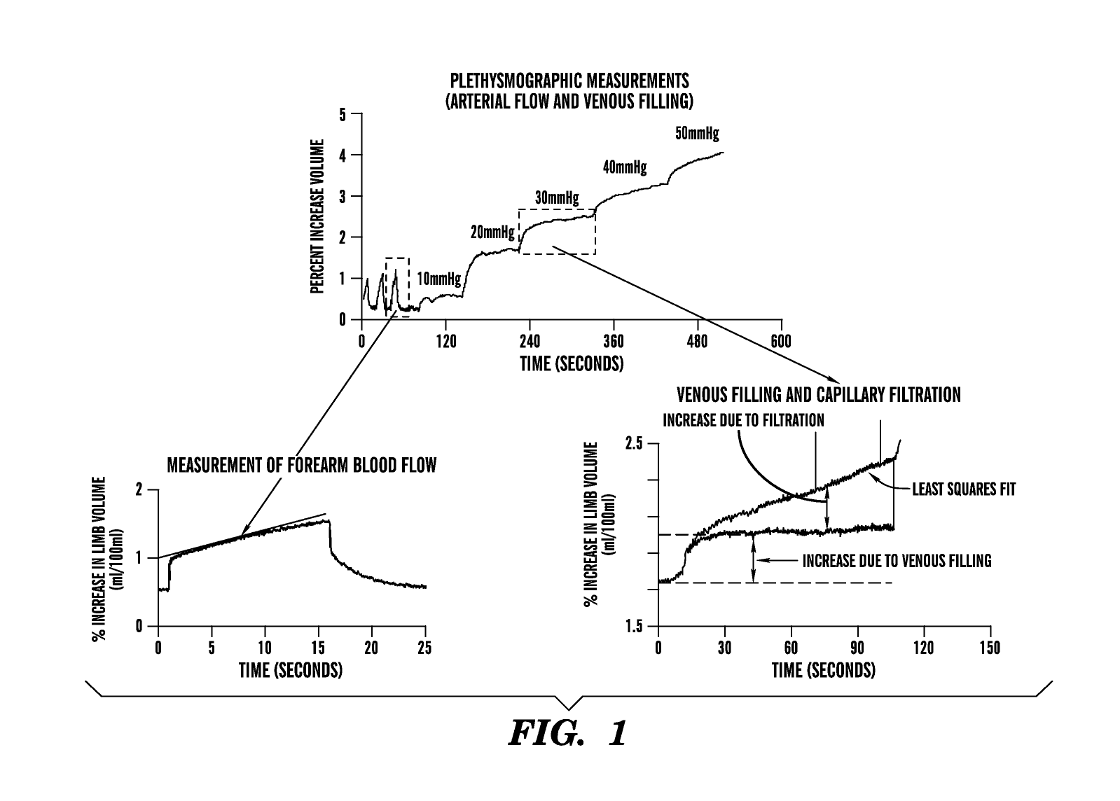

SGP was used to measure calf blood flows, the calf capacitance vessel pressure (venous pressure, denoted Pᵥ), the calf venous volume-pressure capacitance relation, calf venous capacity and the microvascular filtration (flow-pressure) relation in the supine steady state and during steady state upright tilt to 35° in all subjects. Methods were adapted from the work of Gamble et al. (Gamble et al., “Mercury in Silastic Strain Gauge Plethysmography for the Clinical Assessment of the Microcirculation,” Postgrad Med. J., 68 Suppl. 2:S25-S33 (1992); Gamble et al., “The Effect of Passive Tilting on Microvascular Parameters in the Human Calf: A Strain Gauge Plethysmography Study,” J. Physiol. (London) 498 (Pt. 2):541-552 (1997); Gamble et al., “A Reassessment of Mercury in Silastic Strain Gauge Plethysmography for Microvascular Permeability Assessment in Man,” J. Physiol. (London) 464:407-422 (1993), which are hereby incorporated by reference in their entirety), as described, for example, by Stewart et al. (Stewart et al., “Pooling in Chronic Orthostatic Intolerance: Arterial Vasoconstrictive but not Venous Compliance Defects,” Circulation 105:2274-2281 (2002); Stewart et al., “Decreased Skeletal Muscle Pump Activity in Postural Tachycardia Syndrome Patients with Low Peripheral Blood Flow,” Am. J. Physiol. Heart Circ. Physiol. 286:H1216-H1222 (2003), which are hereby incorporated by reference in their entirety), and are summarized in FIGS. 1A, 1B, and 1C.

After a 30-minute resting period, flow measurements were performed in at least triplicate. After returning to baseline, occlusion pressure was increased gradually until limb volume change was just detected. This represents ambient venous pressure denoted Pᵥ. Michel, “Microvascular Permeability, Venous Stasis and Oedema,” Int. Angiol. 8:9-13 (1989), which is hereby incorporated by reference in its entirety. If pressures are applied that are less than Pᵥ, then no increase at all in limb size occurs. The mean arterial pressure (“MAP”) calculated as 0.33*(systolic BP)+0.67*(diastolic BP) and Pᵥ, was used to calculate the calf arterial resistance to blood flow in units of mmHg/(ml/100 ml tissue)/min from

$$\frac{( MAP - {P}_{v} )}{flow} .$$

In order to determine overall calf capacitance the leg was gently raised above heart level until no further decrease in volume was obtained. After recovery, and with the leg flat, 10 mmHg steps in pressure, starting at the first multiple of 10 larger than Pᵥ, to a maximum of 60-70 mmHg, were used, resulting in progressive limb enlargement. Independent data indicate that the venous pressure distal to the congestion cuff approximates the cuff pressure. Christ et al., “Relationship Between Venous Pressure and Tissue Volume During Venous Congestion Plethysmography in Man,” J. Physiol. (London) 503 (Pt. 2):463-467 (1997), which is hereby incorporated by reference in its entirety. Pressure was maintained for 4 minutes in order to reach a steady state.

As shown in FIGS. 1A-1C, at lower congestion pressures the limb size reaches a plateau. With higher pressures, a plateau is not reached (FIG. 1C), but after initial curvilinear changes representing venous filling, the limb continues to increase in size linearly with time for a given pressure step. The linear increase represents microvascular filtration. At a critical pressure greater than Pᵥ, denoted Pᵢ (the isovolumetric pressure of Gamble et al.), the lymphatic system fails to compensate for filtration and the limb interstitium enlarges at a rate in proportion to imposed pressure. This is the pressure threshold for edema formation. At occlusion pressure between Pᵥ and Pᵢ the change in leg size reaches a plateau. At occlusion pressures exceeding Pᵢ, pressure increments result in a change in leg size which is asymptotic to a straight line with positive slope. The singular value decomposition technique (Press et al., “Numerical Recipes in C,” Cambridge UK, Cambridge University Press, pp. 59-70 (1992), which is hereby incorporated by reference in its entirety) was used to fit a least squares straight line to the points comprising the linear microvascular filtration portion of the filling curve at each occlusion pressure, as shown in FIG. 1C. The linear portion is then electronically subtracted from the total curve to obtain a residual curvilinear portion that reaches a plateau. This residual portion is the change of capacitance vessels filling with each pressure step.

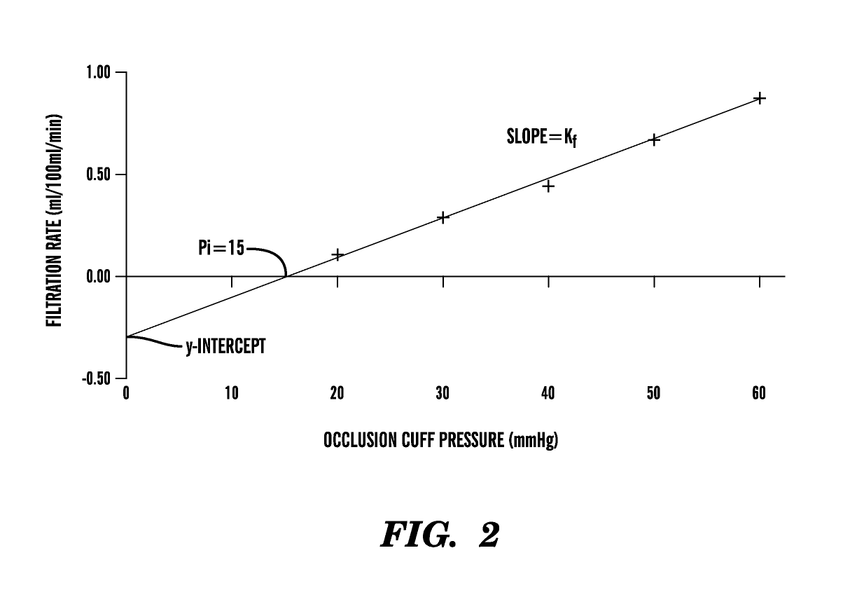

Once the volume response is partitioned, capacitance is calculated from the sum of residual portions, shown as “intravascular filling” in FIGS. 1A-1C, to which is added the estimate of supine venous volume obtained from raising the limb. Stewart et al., “Orthostasis Fails to Produce Active Limb Venoconstriction in Adolescents,” J. Appl. Physiol. 91:1723-1729 (2001), which is hereby incorporated by reference in its entirety. The microvascular filtration relation (filtration rate versus pressure relation) is then constructed for each subject (shown in FIG. 2). Normalized volume is measured and expressed in units of ml volume change/100 ml tissue, normalized filtration rate is expressed in units of ml/100 ml tissue/min, and the normalized filtration coefficient, K_{f} (the slope in the linear relation shown in FIG. 2), is expressed in units of ml/100 ml tissue/min/mmHg. The intercept with the pressure axis of the filtered flow-pressure graph is Pᵢ, at which microvascular filtration exceeds lymphatic flow and approximates the net oncotic pressure gradient for microvascular filtration (discussed further, below). The work of Pappenheimer et al. (Pappenheimer et al, “Effective Osmotic Pressure of the Plasma Proteins and Other Quantities Associated with the Capillary Circulation in the Hindlimbs of Cats and Dogs,” Am. J. Physiol. 152:471-491 (1948), which is hereby incorporated by reference in its entirety) established that net filtration does not occur at pressures less than P_{i.} Thus, the extension of the linear fit to negative flow is a “virtual flow” which serves to estimate the y-intercept with the filtration axis, the normalized filtered flow at zero hydraulic pressure, comprising contributions from lymphatic flow and osmotically driven filtration (discussed further, below).

SPG was used to measure Pᵥ, Pᵢ, the volume-pressure relation of the capacitance vessels and thus overall capacity, and the microvascular filtration relation including the filtration coefficient, K_{f}.

### Example 4

### Peripheral Vascular Evaluation by Impedance Plethysmography (“IGP”)

Impedance plethysmography was used to measure segmental blood flows. Montgomery et al., “An Impedance Device for Study of Multisegment Hemodynamic Changes During Orthostatic Stress,” Aviat. Space Environ. Med. 60:1116-1122 (1989), which is hereby incorporated by reference in its entirety. IPG has also been used to quantify relative body fluid volumes. Gotshall et al., “Bioelectric Impedance as an Index of Thoracic Fluid,” Aviat. Space Environ. Med. 70:58-61 (1999), which is hereby incorporated by reference in its entirety. Relations between impedance and fluid compartmentalization have been established. Geddes et al., “Measurement of Physiological Phenomena by Impedance Changes,” Sem. Med. 124:905-911 (1964), which is hereby incorporated by reference in its entirety. Recently, changes in fluid compartment volumes and transient blood flows have been quantitated during orthostasis. Convertino et al., “Cardiovascular Responses During Orthostasis. Effect of an Increase in VO2max,” Aviat. Space Environ. Med. 55:702-708 (1984); Montgomery et al., “An Impedance Device for Study of Multisegment Hemodynamic Changes During Orthostatic Stress,” Aviat. Space Environ. Med. 60:1116-1122 (1989), which are hereby incorporated by reference in their entirety.

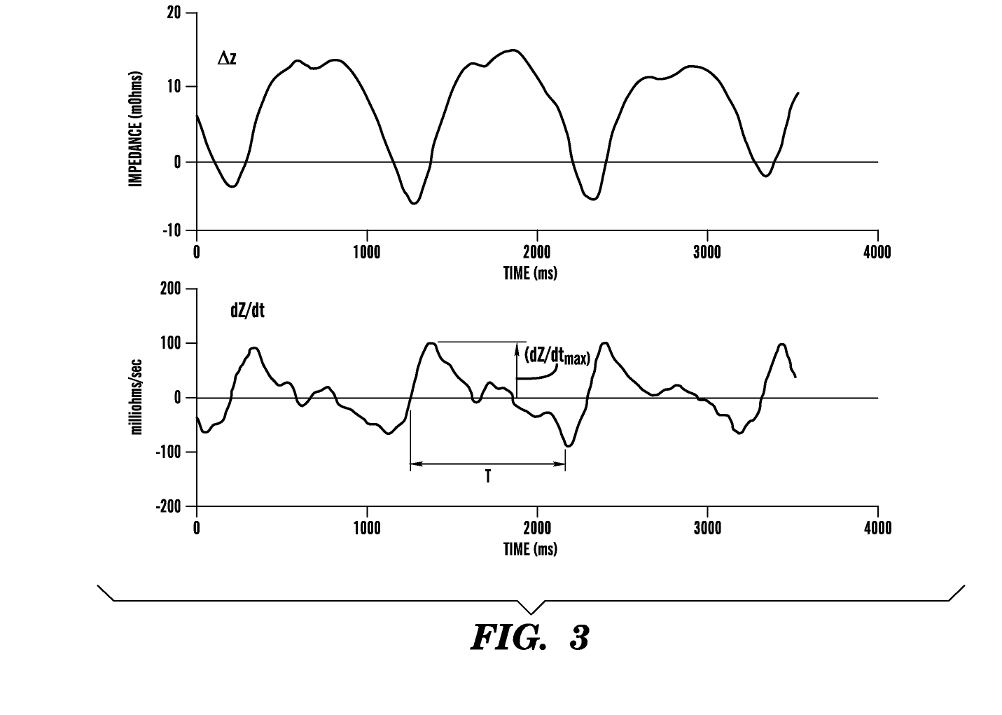

A tetrapolar IPG device was used to measure blood flows in the thoracic segment, splanchnic segment, pelvic-upper leg segment, and calf segment during each test sequence. These segments were demarcated by the location of voltage sampling electrodes as defined previously. Measurements of baseline impedance, Z₀, and pulsatile impedance changes, ΔZ, were made. A high frequency (50 kHz), low amperage (0.1 mA RMS) constant current signal between the foot and hand electrodes was introduced. Z₀ values were measured in each segment continuously. Pulsatile impedance changes were used to compute the time derivative ∂Z/∂t, which was used to obtain the total (ml/min) and relative (ml/100 ml of body tissue/min) blood flow responses of each body segment to each test condition. These results are shown in FIGS. 3A and 3B. Blood flow was estimated for an entire anatomic segment from the formula:

Flow=[HR·ρ·L²·T·∂Z/∂tₘₐₓ]/Z₀²

(Geddes et al., “Detection of Physiological Events by Impedance,” in Principles of Applied Biomedical Instrumentation, New York: Wiley, pp. 594-600 (1989), which is hereby incorporated by reference in its entirety), where HR is heart rate, ρ is the density of blood, L is the distance between the centers of the electrodes, T is the ejection period shown in FIG. 3B, Z is the impedance, and Z₀ is the baseline impedance.

IPG flows are expressed in units of ml/min for an entire anatomic segment. Normalization to tissue volume can be performed.

### Example 5

### 35° Upright Tilt Table Testing

After supine vascular measurements were complete at each vibration frequency, the patients were tilted to 35° for approximately 15 minutes, to obtain steady state circulatory measurements during orthostasis. Earlier work indicated that strain gauge measurements were more accurately determined during 35° compared to 70° upright tilt and that the lesser angle still produces an adequate orthostatic stimulus. Stewart et al., “Contrasting Neurovascular Findings in Chronic Orthostatic Intolerance and Neurocardiogenic Syncope,” Clinical Sci. 104:329-340 (2003), which is hereby incorporated by reference in its entirety. Preliminary studies have shown that this angle of upright tilt can be easily tolerated by all subjects. A quasi-steady state was achieved within approximately 5-7 minutes as previously verified. Heart rate measurement, and arm and leg blood pressures were repeated by oscillometry. Pᵥ was remeasured upright. Limb blood flows were measured at steady state by SGP and IPG. Segmental blood flows were remeasured by IPG.

At steady state, SGP was used to reassess the volume-pressure relation and the microfiltration relation by increasing occlusion cuff pressure beginning at the new measured value of Pv and increasing in 10 mmHg steps up to a maximum pressure less than the diastolic pressure confirmed by oscillometric blood pressure measurement in the contralateral calf. Pᵢ, overall capacity, and K_{f} were obtained by least squares analysis as before. The vertical height between the congestion cuff and the strain gauge was used to correct for hemostatic load differences. Thus, the pressure at the calf strain gauge was adjusted by adding ρ*g*D*sin(35°) where ρ is the density of blood, g is the gravitational acceleration constant and D is the distance from the congestion cuff to the strain gauge.

### Example 6

### Plantar Stimulation

Plantar stimulation was applied using an apparatus consisting of a rectangular shaped frame constructed with an aluminum top plate on which an individual places their feet while in either a supine or upright position. The plate is circumferentially supported by an array of 12 coil springs. Centrally located on the bottom surface of the plate is an electro-mechanical actuator. This actuator is capable of delivering sinusoidal 15-120 Hz vertical displacements of about 0.004-0.24 mm to the top plate. Attached to the underside of the aluminum plate is an accelerometer which provides acceleration feedback to the system. Digital electronic control circuitry automatically adjusted the actuator force to provide an acceleration of 2.0 m/s² (0.2 g p-p). This corresponded to a surface displacement of 240 μm p-p at 15 Hz, and a minimum stimulation amplitude of 25 μm p-p at 45 Hz. The platform was mounted on the footplate of the tilt table throughout the protocol. This is a stable and comfortable arrangement.

### Example 7

### Statistical Data

Tabular data were compared by two-way ANOVA—with vibration frequency, and position (supine and upright) as independent variables. When significant interactions were demonstrated, paired t-tests were used for compared supine and upright changes within-groups. Results are reported as mean±standard deviation. P-values less than 0.05 were considered statistically significant.

Eighteen individuals, ranging in age from 45.5-63.3 years, were recruited for the current study. Patient ages, heights, weights, illnesses, medications, resting blood pressure and heart rate are shown in Table 1. All enrolled subjects were free of acute illnesses. There were no trained competitive athletes. There were no bedridden patients. Informed consent was obtained and all protocols were approved by the Committee for the Protection of Human Subjects (“IRB”) of New York Medical College.

TABLE 1
Patient Data

| | |
|---|---|
| Age (years) | 6 ± 5 |
| Height (cm) | 63 ± 6 |
| Weight (kg) | 1 ± 18 |
| Supine Resting Heart Rate (beats/min) | 7 ± 9 |
| Supine Mean Arterial Pressure (mmHg) | 92 ± 9 |

| | Number of Subjects |
|---|---|
| Illnesses | |
| hypertension | 8 |
| migraine | 3 |
| hypothyroidism | 3 |
| GERD | 3 |
| hypercholesterolemia | 3 |
| asthma | 1 |
| seizure disorder | 1 |
| no illness | 3 |
| Medications | |
| statin drugs | 4 |
| ACE inhibitors | 3 |
| Synthroid ® | 3 |
| beta blockers | 2 |
| selective serotonin reuptake inhibitors | 2 |
| Prevacid ® | 2 |
| SERM | 2 |
| ARB | 1 |
| Tegritol ® | 1 |
| albuterol | 1 |
| inhaled steroid | 1 |
| hydrochlorothiazide | 1 |
| proton pump inhibitor | 1 |

### Example 8

### Heart Rate and Pressure Measurements

As shown in Table 2, heart rate was not affected by the plantar vibration at either frequency, and tended to increase modestly with orthostasis, as expected. Arm mean arterial pressure was unaffected by vibrational frequency or by orthostasis while leg blood pressure increased during tilt as expected due to the hemostatic column imposed by tilting. Leg BP was unaffected by vibrational frequency.

On the other hand, leg venous pressure (Pᵥ) was increased at 15 Hz and at 45 Hz as compared to 0 Hz, while supine (p<0.04), but was not different from 0 Hz when upright. Thus, tilt increased Pᵥ similarly at all stimulus frequencies.

TABLE 2
Hemodynamic Properties

| | | 15 Hz | 45 Hz |
|---|---|---|---|
| | 0 Hz | (% change) | (% change) |
| HR (beats/min) | | | |
| Supine | 65 ± 2 | −1 ± 1 | 0 ± 2 |
| Upright 35° | 72 ± 2† | −3 ± 2 | 0 ± 2† |
| MAP arm (mmHg) | | | |
| Supine | 88 ± 4 | 7 ± 6 | 4 ± 5 |
| Upright 35 | 92 ± 3 | −6 ± 6 | 1 ± 1 |
| MAP leg (mmHg) | | | |
| Supine | 88 ± 4 | 7 ± 6 | 4 ± 5 |
| Upright 35° | 124 ± 4† | −2 ± 2† | 3 ± 2† |
| Pv leg (mmHg) | | | |
| Supine | 13 ± 1 | 14 ± 7* | 16 ± 6* |
| Upright 35° | 27 ± 2† | −1 ± 2† | 3 ± 2† |
| SGP leg arterial resistance | | | |
| (mmHg/ml/100 ml/min) | | | |
| Supine | 41 ± 7 | −6 ± 7 | −4 ± 5 |
| Upright 35° | 126 ± 28 | 11 ± 15 | 7 ± 9 |
| SGP leg venous resistance | | | |
| (mmHg/ml/100 ml/min) | | | |
| Supine | 1.3 ± .4 | 8 ± 12 | −10 ± 10 |
| Upright 35° | 1.2 ± .4 | 11 ± 40 | −27 ± 12*† |
| IPG cal segmental flow | | | |
| (ml/100 ml/min) | | | |
| Supine | 137 ± 18 | 10 ± 4* | 30 ± 7* |
| Upright 35° | 99 ± 15† | 32 ± 12*† | 47 ± 14*† |
| IPG pelvic segmental | | | |
| flow (ml/mim) | | | |
| Supine | 707 ± 90 | 31 ± 15 | 26 ± 9* |
| Upright 35° | 713 ± 80 | 22 ± 15 | 35 ± 11* |
| IPG visceral segmental | | | |
| flow (ml/min) | | | |
| Supine | 1906 ± 253 | 6 ± 9 | 11 ± 14 |
| Upright 35° | 1288 ± 302† | 0 ± 15† | 7 ± 14† |
| IPG thorax segmental | | | |
| flow (ml/min) | | | |
| Supine | 3506 ± 322 | 10 ± 5* | 20 ± 7* |
| Upright 35° | 2688 ± 287† | 16 ± 8† | 17 ± 7*† |

* = p < .05 compared to 0 Hz
† = p < .05 compared to supine

### Example 9

### Peripheral Blood Flow and Resistance Measurements

As shown in Table 2, supine blood pressure, as well as arterial resistance and venous resistance as measured by strain gauge plethysmography, were unaffected by plantar stimulation. As expected, blood flow decreased while peripheral arterial resistance increased with upright tilt. There was no effect of plantar stimulation on arterial resistance when supine or upright. Venous resistance was unaffected by orthostasis, but decreased during 45 Hz plantar stimulation in the upright position (from 1.2±0.2 at 0 Hz, to 1.2±0.5 at 15 Hz, and to 0.7±0.1 mmHg/ml/100 ml/min, p<0.05).

IPG measurements showed that calf segmental blood flow was significantly affected both by orthostasis and plantar stimulation. In the supine position, calf flow increased from 137±18 ml/min, to 150 ml/min (p=0.05) at 15 Hz, to 178 ml/min (p=0.05) at 45 Hz. Orthostasis resulted in a significant reduction in calf flow to 99±15 ml/min (p=0.05 compared to supine). However, plantar stimulation at 15 Hz increased calf flow to 131 ml/min (p=0.005) and to 146 ml/min with 45 Hz stimulation (p=0.001).

As shown in Table 2, upper leg-pelvic blood flows were unaffected by orthostasis but increased during plantar stimulation while supine. During upright tilt, plantar vibration increased pelvic flow from 713 at 0 Hz, to 869 at 15 Hz, and finally to 963 ml/min, at 45 Hz, p<0.005). Splanchnic flow was unaffected by the plantar vibration, but decreased during orthostasis at all frequencies (p<0.001).

Thoracic blood flow decreased as expected with orthostasis (p<0.05) and was increased to a similar extent in the supine and upright positions by plantar stimulation (from 3506±322 at 0 Hz, to 3990±270 at 15 Hz, and finally to 4237±366 ml/min at 45 Hz, p<0.02 when supine and from 2688±287 at 0 Hz, to 3391±688 at 15 Hz, and finally to 3670±313 ml/min at 40 Hz, p<0.02) when upright.

### Example 10

### Volume-Pressure Capacitance And Microfiltration Relations

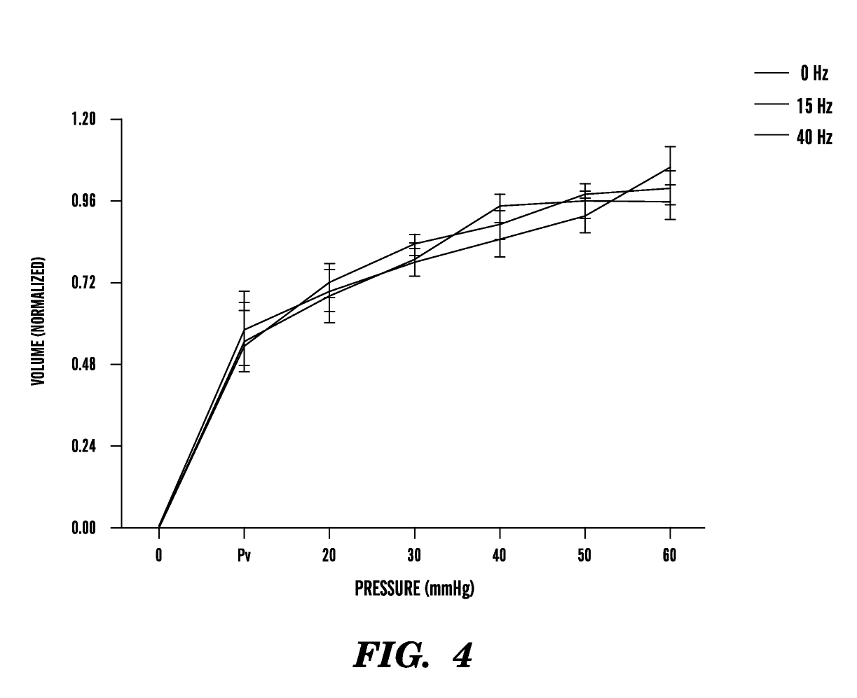

The volume-pressure relation as depicted in FIG. 4 is unaffected by plantar stimulation, and as shown previously (Stewart et al., “Orthostasis Fails to Produce Active Limb Venoconstriction in Adolescents,” J. Appl. Physiol. 91:1723-1729 (2001), which is hereby incorporated by reference in its entirety), is unaffected by orthostasis. Thus, the maximum leg capacity (the upper limit of pressure generated volume increase) was unaffected by tilt or stimulation.

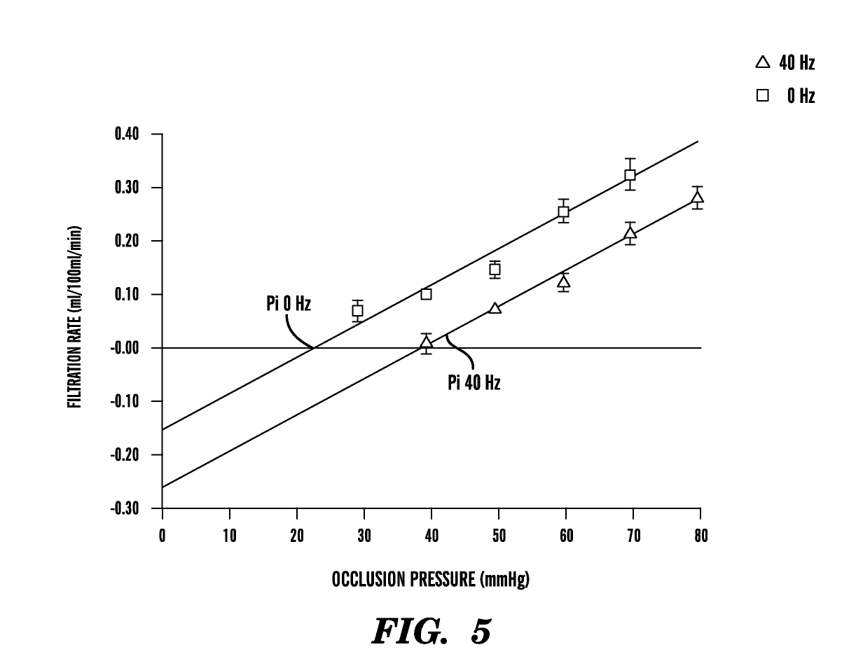

As shown in FIG. 5, however, the microvascular filtration relation is shifted rightwards with plantar stimulation and is unaffected by upright tilt. There is no change in the slope of the relation, K_{f}, with vibrational frequency but a pronounced shift in x-intercept (Pᵢ) and y intercept. Thus, while supine, Pᵢ increases from 24±2 at 0 Hz, to 27±3 at 15 Hz, and to 31±2 mmHg at 45 Hz, (p<0.01), and while upright, Pᵢ increases from 25±3 at 0 Hz, to 28±4 at 15 Hz, and to 35±4 mmHg at 45 Hz, (p<0.04).

The most substantial effect of plantar stimulation on circulation in this study was on calf blood flow, which is significantly decreased by orthostasis, but completely normalized with plantar vibration. As importantly, 45 Hz plantar vibration significantly enhanced calf blood flow even when the subjects were in the supine position. As supine subjects experience essentially no mechanical loading of their musculo-skeletal system, this indicates that cutaneous receptors on the plantar surface are responsible for the observed effects.

Similarly, the significant changes in upper leg-pelvic blood flow and thoracic flow due to orthostasis are eliminated or blunted by plantar stimulation, in particular, with stimulation at 45 Hz. The changes in leg-pelvic flow are produced as part of a generalized lower extremity effect affecting calf and thigh alike, and the increase in thoracic IPG flow represents the increase in overall systemic flow due to improved peripheral flow and venous return stimulated by the plantar vibration. The results indicate that while microvascular filtration per se is unaffected by plantar vibration, there is enhanced lymphatic and venous drainage particularly evident when upright.

Another significant finding is that the microvascular filtration relation is right-shifted by plantar vibration as a consequence of an increase in Pᵢ, the threshold for edema, while the microvascular filtration coefficient, K_{f}, remains unchanged. This occurs in both the supine and upright position and to similar degree. While supine, venous pressure is slightly increased by plantar vibration, the volume-pressure relation is unchanged and thus the capacitance relation is unchanged.

Previous work by Zweifach and others (Zweifach et al., “Fluid Exchange Across the Blood Capillary Interface,” Fed. Proc. 25:1784-1788 (1966), which is hereby incorporated by reference in its entirety) indicate that under normal conditions capillary flow is unidirectional—predominantly from vessel lumen to interstitium—with lymphatic drainage removing filtered fluid. There is no sustained reabsorption of interstitial fluid at low capillary pressure. Christ et al., “Relationship Between Venous Pressure and Tissue Volume During Venous Congestion Plethysmography in Man,” J. Physiol. (London) 503 (Pt. 2):463-467 (1997); Michel, “Starling: The Formulation of His Hypothesis of Microvascular Fluid Exchange and Its Significance After 100 Years,” Exp. Physiol. 82:1-30 (1997), which are hereby incorporated by reference in their entirety. Also, changes in plasma oncotic pressure are small (on the order of 3%) while traversing the capillary bed at ordinary flow rates. Zweifach, “Microcirculation,” Annu. Rev. Physiol. 35:117-150 (1973), which is hereby incorporated by reference in its entirety.

The Landis-Starling relation implies that:

Filtration=K_{f}·[(P_{vasc}−Pₜ)−σ(Π_{vasc}−Πₜ)]

(Landis, “Microinjection Studies of Capillary Permeability II: The Relation Between Capillary Pressure and the Rate at Which Fluid Passes Through the Walls of Single Capillaries,” Am. J. Physiol. 82:217-238 (1927), which is hereby incorporated by reference in its entirety), where P_{vasc} is the vascular pressure, Pₜ the tissue pressure, Π_{vasc} and Πₜ are corresponding oncotic pressures, and σ is the [protein] reflection coefficient. The net increase in limb tissue volume due to filtration is therefore equal to the filtered fluid minus the lymphatic drainage. Provided lymphatic drainage remains adequate there is no extra collection of interstitial fluid, i.e. no edema. When fluid filtration is low it is balanced by lymph flow. This prevails at pressures less than Pᵢ, defined here as the threshold for edema formation. Michel, “Microvascular Permeability, Venous Stasis and Oedema,” Int. Angiol. 8:9-13 (1989), which is hereby incorporated by reference in its entirety. When filtered flow exceeds lymphatic flow, edema results. The mass balance relation becomes:

$$\frac{d {Vol}_{t}}{d t} = {K}_{f} \cdot [ ( {P}_{vasc} - {P}_{t} ) - σ ( {\prod }_{vasc} - {\prod }_{t} ) ] - Lymphatic drainage .$$

During small pressure steps such as employed here there is no change in blood flow and a small decrement in precapillary sphincter resistance. Gamble et al., “Human Calf Precapillary Resistance Decreases in Response to Small Cumulative Increases in Venous congestion Pressure,” J. Physiol. (London) 507 (Pt. 2):611-617 (1998), which is hereby incorporated by reference in its entirety. Tissue pressure is also small, and assuming tissue pressure, reflection coefficient, and oncotic pressures are relatively unchanged during cuff occlusion, the rate of change in limb volume, dV/dt, is a function of P_{vasc} and lymphatic drainage, where P_{vasc} is determined by venous occlusion pressure. Most generally one expects lymphatic drainage to change as P_{vasc} changes.

$$\frac{d {Vol}_{t}}{d t}$$

as a function of P_{vasc} defines the microvascular filtration relation which, as measured, is linear in P_{vasc} alone. This has two implications. First, lymphatic drainage is independent of time and either constant (zero order) or changes linearly (first order) in P_{vasc}; Olszewski et al. (Olszewski et al., “Lymph Flow and Protein in the Normal Male Leg During Lying, Getting Up, and Walking,” Lymphology 10:178-183 (1977), which is hereby incorporated by reference in its entirety) and Michel et al. (Michel et al., “The Measurement of Fluid Filtration in Human Limbs,” in, Clinical Investigations of Microcirculation Tooke et al., ed., Boston: L H Morhuis Nyhoff, pp. 103-126 (1987), which is hereby incorporated by reference in its entirety) demonstrated constant lymphatic pumping capability with increased venous pressure making a zero order process likely. Michel, “Microvascular Permeability, Venous Stasis and Oedema,” Int. Angiol. 8:9-13 (1989), which is hereby incorporated by reference in its entirety.

Secondly, at the x-intercept,

$${P}_{vasc} = {P}_{i} , \frac{d {Vol}_{t}}{d t} = 0$$

K_{f}·[(Pᵢ−Pₜ)−σ(Π_{vasc}−Πₜ)]=Lymphatic drainage

Lymphatic drainage=K_{f}·Pᵢ−K_{f}·Pₜ−σ(Π_{vasc}−Πₜ)]≈K_{f}·Pᵢ−constant (+perhaps a small variable term), and the y intercept (P_{vasc}=0 in FIG. 2) relates to the lymphatic drainage by the formula:

Y_{intercept}=K_{f}·[(−Pₜ)−σ(Π_{vasc}−Πₜ)]−Lymphatic drainage≈K_{f}·Pᵢ.

The present study shows that P, increases with plantar stimulation and that Pₜ is small. A more negative Y-intercept therefore implies increased lymphatic drainage in subjects at the higher (45 Hz) plantar stimulation frequency.

Lymph is formed by the translocation of interstitial fluid into the initial lymphatics by osmotic or vesicular transport mechanisms. Aukland et al., “Interstitial-Lymphatic Mechanisms in the Control of Extracellular Fluid Volume,” Physiol. Rev. 73:1-78 (1993), which is hereby incorporated by reference in its entirety. There is probably a small facilitating pressure gradient from interstitium to lymphatic which may be produced by suction effects. Guyton et al., “The Energetics of Lymph Formation,” Lymphology 13:173-176 (1980), which is hereby incorporated by reference in its entirety. The initial lymphatics may possess active contractile activity using actin filaments and there are valve-like structures aiding centripetal flow, although true valves are not present. Muthuchamy et al., “Molecular and Functional Analyses of the Contractile Apparatus in Lymphatic Muscle,” FASEB J. 17:920-922 (2003), which is hereby incorporated by reference in its entirety. Transport from initial lymphatics to valve containing lymphatic ducts remains controversial but seems to depend on tissue movement: thus, chronically immobilized tissues have almost no lymphatic flow especially in the extremities. Gnepp et al., “The Effect of Passive Motion on the Flow and Formation of Lymph,” Lymphology 11:32-36 (1978), which is hereby incorporated by reference in its entirety. Unlike veins, there is apparently little effect of “force from behind” (cardiac muscle and blood pressure) on lymphatic fluid propulsion. Instead, lymph flow is enhanced by active and passive limb muscle movements, the skeletal muscle pump. Reddy, “Lymph Circulation: Physiology, Pharmacology, and Biomechanics,” Crit. Rev. Biomed. Eng. 14:45-91 (1986), which is hereby incorporated by reference in its entirety. Prior work indicates that to create unidirectional flow, these external forces must be intermittent. McGeown et al., “The Role of External Compression and Movement in Lymph Propulsion in the Sheep Hind Limb,” J. Physiol. 387:83-93 (1987), which is hereby incorporated by reference in its entirety. Examples of external factors producing lymphatic flow include respiration, peristaltic action (in the mesentery), passive and active limb movements through the actions of postural musculature, and compression by external agencies (e.g. massage). In the subjects of the present study, there is no particular alteration in external force and therefore, enhanced lymphatic flow results from limb muscle movement experimentally stimulated by plantar vibration.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of enhancing blood and lymph flow in a human subject comprising:
selecting a human subject in need of enhanced blood and lymph flow and
applying an electrical stimulus to one or more plantar and palmer surfaces in the selected subject, said stimulus effective to directly stimulate cutaneous receptors in said plantar and palmer surfaces, thereby enhancing blood and lymph flow in the selected subject.

2. The method according to claim 1, wherein the electrical stimulus is in the frequency range of 30-60 Hz.

3. The method according to claim 1, wherein the electrical stimulus is about 45 Hz.

4. The method according to claim 1, wherein the electrical stimulus induces an electric field in the plantar and palmer surfaces in the range of 0.1-10 V/m.

5. The method according to claim 1, wherein said method is used to treat a condition selected from the group consisting of edema, poor wound healing, lymphedema, orthostatic intolerance (hypotension when upright), chronic fatigue syndrome, lower back pain, fuzzy vision, poor concentration due to hypotension, loss of balance/dizziness/fainting, deep vein thrombosis, pulmonary embolism, skin ulcers, pressure sores, bone loss/osteoporosis, and blood flow related complications of diabetes.

6. The method according to claim 1, wherein the subject is in a non-upright position during said applying the electrical stimulus.

7. The method according to claim 6, wherein the subject is in a supine position during said applying of the electrical stimulus.

8. A method of enhancing blood and lymph flow in a human subject comprising:
selecting a human subject in need of enhanced blood and lymph flow and
applying an electrical stimulus to one or more plantar surfaces in a subject, said stimulus effective to directly stimulate cutaneous receptors in said plantar surfaces, thereby enhancing blood and lymph flow in the selected human subject.

9. The method according to claim 8, wherein the electrical stimulus is in the frequency range of 30-60 Hz.

10. The method according to claim 9, wherein the electrical stimulus is about 45 Hz.

11. The method according to claim 8, wherein the electrical stimulus induces an electric field in the plantar surfaces in the range of 0.1-10 V/m.

12. The method according to claim 8, wherein said method is used to treat a condition selected from the group consisting of edema, poor wound healing, lymphedema, orthostatic intolerance (hypotension when upright), chronic fatigue syndrome, lower back pain, fuzzy vision, poor concentration due to hypotension, loss of balance/dizziness/fainting, deep vein thrombosis, pulmonary embolism, skin ulcers, pressure sores, bone loss/osteoporosis, and blood flow related complications of diabetes.

13. The method according to claim 8, wherein the subject is in a non-upright position during said applying the electrical stimulus.

14. The method according to claim 13, wherein the subject is in a supine position during said applying of the electrical stimulus.

15. A method of enhancing blood and lymph flow in a human subject comprising:
selecting a human subject in need of enhanced blood and lymph flow and
applying an electrical stimulus to one or more palmer surfaces in the selected subject, said stimulus effective to directly stimulate cutaneous receptors in said palmer surface, thereby enhancing blood and lymph flow in the selected subject.

16. The method according to claim 15, wherein the electrical stimulus is in the frequency range of 30-60 Hz.

17. The method according to claim 16, wherein the electrical stimulus is about 45 Hz.

18. The method according to claim 15, wherein the electrical stimulus induces an electric field in the palmer surfaces in the range of 0.1-10 V/m.

19. The method according to claim 15, wherein said method is used to treat a condition selected from the group consisting of edema, poor wound healing, lymphedema, orthostatic intolerance (hypotension when upright), chronic fatigue syndrome, lower back pain, fuzzy vision, poor concentration due to hypotension, loss of balance/dizziness/fainting, deep vein thrombosis, pulmonary embolism, skin ulcers, pressure sores, bone loss/osteoporosis, and blood flow related complications of diabetes.

20. The method according to claim 15, wherein the subject is in a non-upright position during said applying the electrical stimulus.

21. The method according to claim 20, wherein the subject is in a supine position during said applying of the electrical stimulus.
